(19) United States
(12) Patent Application Publication
(10) Pub. No.: US 2008/0114358 A1
(43) Pub. Date: May 15, 2008

(54) Intervertebral Prosthetic Assembly for Spinal Stabilization and Method of Implanting Same

(75) Inventors: Kent M. Anderson, Memphis, TN (US); Eric C. Lange, Collierville, TN (US); Jean Taylor, Cannes (FR)

Correspondence Address: HAYNES AND BOONE, LLP, 901 Main Street, Suite 3100, Dallas, TX, 75202, US

(73) Assignee: Warsaw Orthopedic, Inc., Warsaw, IN (US)

(21) Appl. No.: 11/559,013

(22) Filed: Nov. 13, 2006

(51) Int. Cl. A61B 17/58; A61F 2/44; A61B 17/08

(52) U.S. Cl. 606/64; 623/17.16; 606/61; 606/151

(57) ABSTRACT

A prosthetic assembly and method of implanting same, according to which a least one cross-bar is secured to the spinal column. A spacer engages the spinous process of a vertebra of the spinal column. The cross-bar is connected to the spacer via an adapter.

### BACKGROUND

The present invention relates to an intervertebral prosthetic assembly for stabilizing the human spine, and a method of implanting same.

Intervertebral discs that extend between adjacent vertebrae in vertebral columns of the human body provide critical support between the adjacent vertebrae while permitting multiple degrees of motion. These discs can rupture, degenerate, and/or protrude by injury, degradation, disease, or the like, to such a degree that the intervertebral space between adjacent vertebrae collapses as the disc loses at least a part of its support function, which can cause impingement of the nerve roots and severe pain.

Intervertebral prosthetic devices have been designed that can be implanted between the adjacent vertebrae, both anterior and posterior of the column. Many of these devices are supported between the spinous processes of the adjacent vertebrae to prevent the collapse of the intervertebral space between the adjacent vertebrae and provide motion stabilization of the spine.

However, in some cases it is often necessary to perform a laminectomy to remove the laminae and the spinous process from at least one vertebra to remove an intervertebral disc and/or to decompress a nerve root. Typically, in these procedures, two vertebral segments are fused together to stop any motion between the segments and thus relieve the pain. In this situation, it would be impossible to implant an intervertebral prosthetic device of the above type since the device requires support from the respective spinous processes of both adjacent vertebrae.

The present invention is thus directed to an intervertebral prosthetic assembly that is implantable between two adjacent vertebrae to provide motion stabilization, despite the fact that at least one vertebra is void of a spinous process. Various embodiments of the invention may possess one or more of the above features and advantages, or provide one or more solutions to the above problems existing in the prior art.

### BRIEF DESCRIPTION OF THE DRAWINGS

FIG. 1 is a side elevational view of an adult human vertebral column.

FIG. 2 is a posterior elevational view of the column of FIG. 1.

FIG. 3 is an enlarged, front elevational view of one of the vertebrae of the column of FIGS. 1 and 2.

FIG. 4 is an isometric view of a portion of the column of FIGS. 1 and 2, including the lower three vertebrae of the column, and depicting an intervertebral prosthetic assembly according to an embodiment of the invention implanted between two adjacent vertebrae.

FIG. 5 is an enlarged view of a portion of the column and the assembly shown in FIG. 4.

FIGS. 6 and 7 are views similar to that of FIG. 5, but depicting alternate embodiments of the assembly of FIG. 5.

FIGS. 8 and 9 are partial elevational/partial sectional views of two additional alternate embodiments of the assembly of FIG. 5.

### DETAILED DESCRIPTION

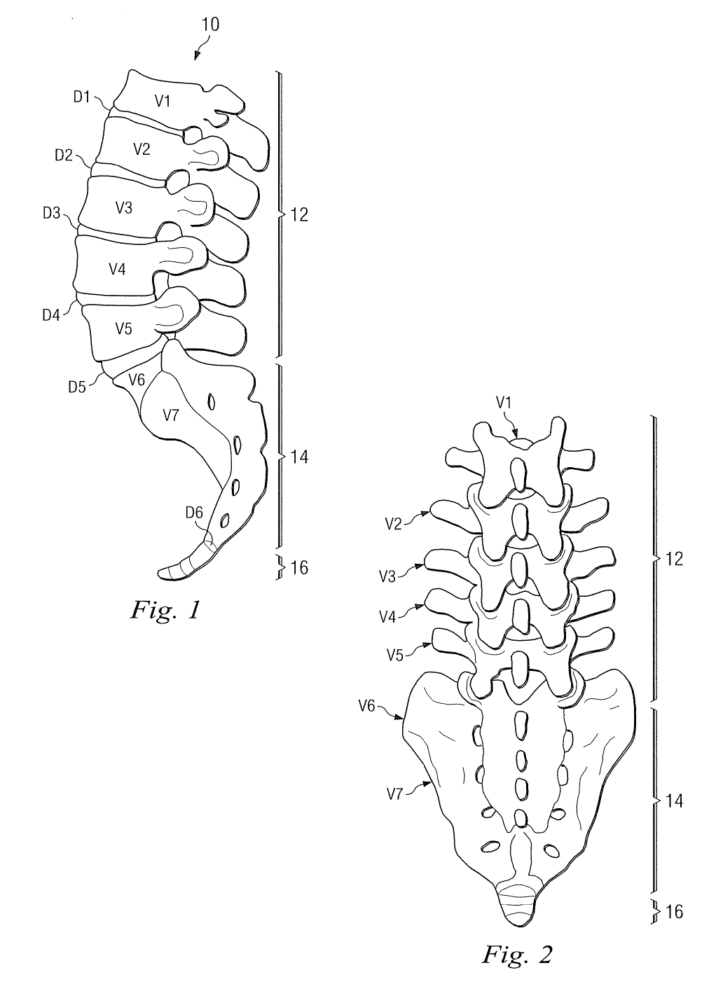

With reference to FIGS. 1 and 2, the reference numeral 10 refers, in general, to the lower portion of a human vertebral column. The column 10 includes a lumbar region 12, a sacrum 14, and a coccyx 16. The flexible, soft portion of the column 10, which includes the thoracic region and the cervical region, is not shown.

The lumbar region 12 of the vertebral column 10 includes five vertebrae V1, V2, V3, V4 and V5 separated by intervertebral discs D1, D2, D3, and D4, with the disc D1 extending between the vertebrae V1 and V2, the disc D2 extending between the vertebrae V2 and V3, the disc D3 extending between the vertebrae V3 and V4, and the disc D4 extending between the vertebrae V4 and V5.

The sacrum 14 includes five fused vertebrae, one of which is a superior vertebra V6 separated from the vertebra V5 by a disc D5. The other four fused vertebrae of the sacrum 14 are referred to collectively as V7. A disc D6 separates the sacrum 14 from the coccyx 16, which includes four fused vertebrae (not referenced).

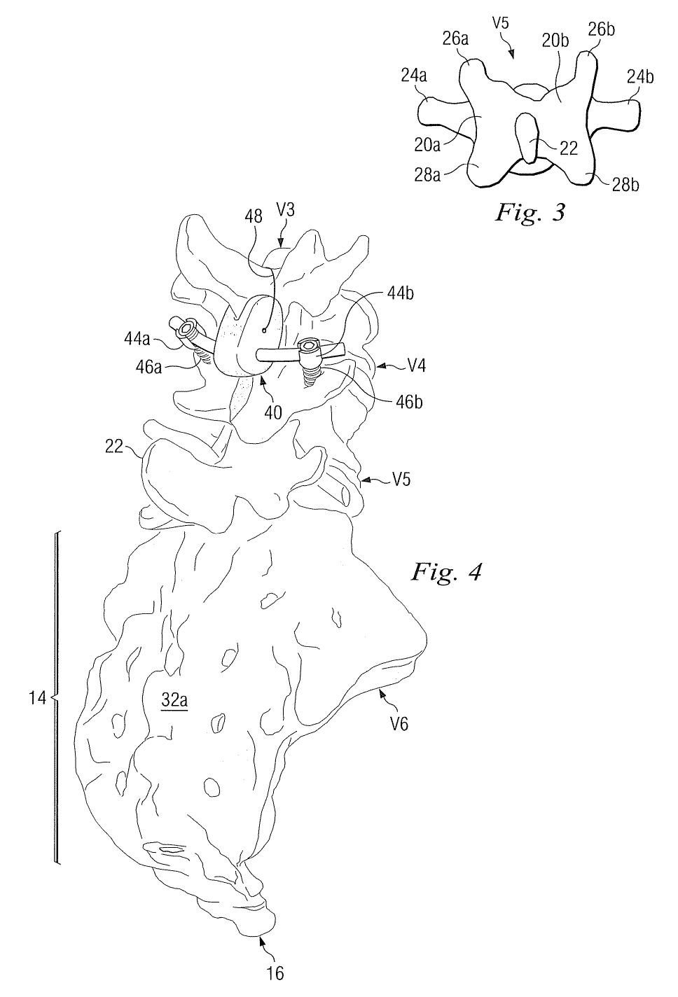

With reference to FIG. 3, the vertebra V5 includes two laminae 20a and 20b extending to either side (as viewed in FIG. 2) of a spinous process 22 that extends posteriorly from the juncture of the two laminae. Two transverse processes 24a and 24b extend laterally from the laminae 20a and 20b, respectively. Two articular processes 26a and 26b extend superiorly from the laminae 20a and 20b respectively, and two articular processes 28a and 28b extend inferiorly from the laminae 20a and 20b, respectively. The inferior articular processes 28a and 28b rest in the superior articular process of the vertebra V2 to form a facet joint. Since the vertebrae V1-V4 are similar to the vertebra V5, and since the vertebrae V6 and V7 are not involved in the present invention, they will not be described in detail.

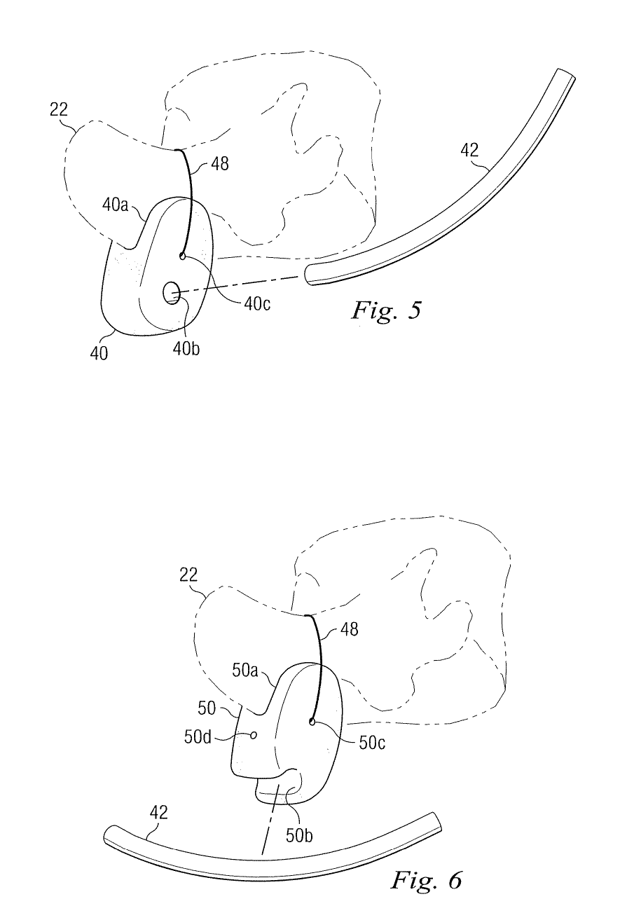

Referring to FIGS. 4 and 5 it will be assumed that, for one or more of the reasons set forth above, the spinous process 22 of V4 has been removed, the vertebrae V3, V4, and/or V5 are not being adequately supported by the discs D3 and/or D4, and that it is desired to provide supplemental support and motion stabilization for these vertebrae.

To this end, a spacer 40 is provided that is fabricated from a relatively flexible, soft material, and is substantially rectangular in shape with the exception that a curved notch, or saddle, 40a is formed at one end for receiving the spinous process 22 of the vertebra V3.

A through opening 40b extends through the spacer in a spaced relation to the saddle 40a, and a flexible cross-bar 42 extends through the opening 40b in the spacer 40 and generally transverse to the axis of the spine. The cross-bar 42 spans a substantial portion of the width of the vertebra V4.

Two transversely-spaced retainers 44a and 44b (FIG. 4) are fastened to the vertebra V4 by two screws 46a and 46b, respectively. Each screw 46a and 46b has a head (not shown) extending in a corresponding retainer, and an externally threaded shank extending from the head that is screwed in the vertebra V4. The respective end portions of the cross-bar 42 extend through openings in the retainers 44a and 44b.

A strap 48 extends through another opening 40c in the spacer 40 and around the process 22 of the vertebra V3 to secure the spacer to the process.

The spacer 40 is thus firmly secured in its implanted position shown in FIG. 4, and stabilizes the vertebrae V3-V5. Also, the relatively flexible, soft spacer 40 readily conforms to the process 22 of the vertebra V3 and provides excellent shock absorption and deformability, resulting in an improved fit.

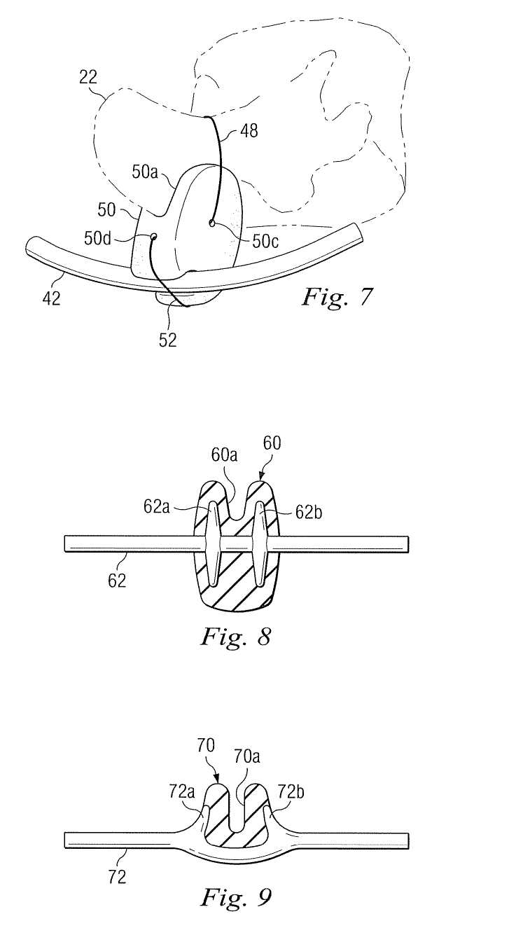

The embodiment of FIGS. 6 and 7 is similar to that of FIGS. 4 and 5 and identical components are given the same reference numerals. According to the embodiment of FIGS. 6 and 7, a spacer 50 is provided that is fabricated from a relatively flexible, soft material, and is substantially rectangular in shape with the exception that a saddle 50a is formed at one end of the spacer for receiving the spinous process 22 of the vertebra V3. Also, a transversely extending notch, or groove 50b is formed in the other end of the spacer 50, and two through openings 50c and 50d extend through the spacer, for reasons to be described.

A central portion of the cross-bar 42 of the previous embodiment extends into the notch 50b and generally transverse to the axis of the spine, and spans a substantial portion of the width of the vertebra V4. As in the previous embodiment, the respective end portions of the cross-bar 42 extend through openings in the retainers 44a and 44b (FIG. 4) which are mounted to the vertebra V4 by the screws 46a and 46b, respectively. The strap 48 extends through the opening 50c in the spacer 50 and around the process 22 of the vertebra V3 to secure the spacer to the vertebra. According to the embodiment of FIGS. 6 and 7, a second strap 52 (FIG. 7) extends through the opening 50d in the spacer 50 and around the notch 50b and the cross-bar 42, to secure the cross-bar to the spacer.

The spacer 50 is thus firmly secured in the same implanted position as shown in connection with the spacer 40 of the embodiment of FIGS. 4 and 5, and stabilizes the vertebrae V3-V5. Also, the relatively flexible, soft, spacer 50 readily conforms to the process 22 of the vertebra V3 and provides excellent shock absorption and deformability resulting in an improved fit.

The embodiment of FIG. 8 is similar to that of the embodiments and FIGS. 4 and 5 and identical components are given the same reference numerals. According to the embodiment of FIG. 8, a spacer 60 is provided that is fabricated from a relatively flexible, soft material, and is substantially rectangular in shape with the exception that a saddle 60a is formed at one end for receiving the spinous process 22 of the vertebra V3.

A flexible cross-bar 62 is provided that has two slightly-spaced, circular flanges 62a and 62b formed on its central portion. The central portion of the cross-bar 62, along with the flanges 62a and 62b are embedded in the spacer 60 in any conventional manner, such as by forming the spacer of a rubber material and molding it over the cross-bar.

As in the previous embodiments, the respective end portions of the cross-bar 62 extend through openings in the retainers 44a and 44b (FIG. 4), which are mounted to the vertebra V4 by the screws 46a and 46b, respectively, as described above. Also, although not shown in FIG. 8, it is understood that the strap 48 of the embodiment of FIGS. 4 and 5 can extend through the spacer 60 and around the process 22 of the vertebra V3 to secure the spacer to the vertebra.

The spacer 60 is thus firmly secured in the same implanted position as shown in connection with the spacer 40 of the embodiment of FIGS. 4 and 5, and stabilizes the vertebrae V3-V5. Also, the relatively flexible, soft spacer 60 readily conforms to the process 22 of the vertebra V3 and provides excellent shock absorption and deformability resulting in an improved fit.

The embodiment of FIG. 9 is similar to that of FIGS. 4-8 and identical components are given the same reference numerals. According to the embodiment of FIG. 9 a spacer 70 is provided that is fabricated from a relatively flexible, soft material, and has a generally U-shaped cross section. A saddle 70a is defined at one end of the spacer 70 for receiving the spinous process 22 of the vertebra V3.

A flexible cross-bar 72 is provided that has two slightly-spaced protrusions 72a and 72b that extend transverse to the axis of the cross-bar and form, with the corresponding portion of the cross-bar, a U-shaped portion that receives the spacer 70. In this context, the spacer 70 could be formed of a rubber material that is molded over the cross-bar 72.

As in the previous embodiments, the respective end portions of the cross-bar 72 extend through openings in the retainers 44a and 44b (FIG. 4), which are mounted on the vertebra V4 by the screws 46a and 46b in the manner described above. Also, although not shown in FIG. 9, it is understood that the strap 48 of the embodiment of FIGS. 4 and 5 can extend through the spacer 70 and around the process 22 of the vertebra V3 to secure the spacer to the vertebra.

The spacer 70 is thus firmly secured in the same implanted position as shown in connection with the spacer 40 of the embodiment of FIGS. 4 and 5, and therefore stabilizes the vertebrae V3-V5. Also, the relatively flexible, soft spacer 70 readily conforms to the process 22 of the vertebra V3 and provides excellent shock absorption deformability resulting in an improved fit.

### Variations

It is understood that variations may be made in the foregoing without departing from the invention and examples of some variations are as follows:

(1) The assemblies of the above embodiments can be inserted between two vertebrae following a discectemy in which a disc between the adjacent vertebrae is removed, or corpectomy in which at least one vertebrae is removed.

(2) The cross-bars in each of the previous embodiments can be rigidly connected to the pedicles of the vertebra by means other than the screws and retainers described in the above examples.

(3) The components disclosed above can be fabricated from materials other than those described above and may include a combination of soft and rigid materials.

(4) Any conventional substance that promotes bone growth, such as HA coating, BMP, or the like, can be incorporated in the spacers in the above embodiments.

(5) The surfaces of the spacers disclosed above that define the saddles that receive the spinous process can be treated, such as by providing teeth, ridges, knurling, etc., to better grip the spinous process.

(6) The spacers disclosed above can be fabricated of a permanently deformable material thus providing a clamping action against the spinous processes.

(7) One or more of the components disclosed above may have through-holes formed therein to improve integration of the bone growth.

(8) The components of one or more of the above embodiments may vary in shape, size, composition, and physical properties.

(9) Through-openings can be provided through one or more components of each of the above embodiments to receive tethers for attaching the devices to a vertebra or to a spinous process.

(10) The assemblies of each of the above embodiments can be placed between two vertebrae in the vertebral column other than the ones described above.

(11) The number and lengths of the cross-bars in one or more of the embodiments can be varied.

(12) The cross-bars can be flexible or rigid.

(13) The assemblies of the above embodiments can be implanted between body portions, or anatomical structures other than vertebrae.

(14) The spatial references made above, such as “under”, “over”, “between”, “flexible, soft”, “lower”, “top”, “bottom”, “axial”, “transverse”, etc. are for the purpose of illustration only and do not limit the specific orientation or location of the structure described above.

The preceding specific embodiments are illustrative of the practice of the invention. It is to be understood, therefore, that other expedients known to those skilled in the art or disclosed herein, may be employed without departing from the invention or the scope of the appended claims, as detailed above. In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

What is claimed is:

1. An assembly for insertion between two anatomical structures, the device comprising:
a spacer engaging one of the structures;
a device connecting the spacer to the one structure; and
at least one cross-bar secured to the spacer and to one of the structures.

2. The assembly of claim 1 wherein the device is a strap.

3. The assembly of claim 1 wherein the cross-bar extends through an opening in the spacer.

4. The assembly of claim 1 wherein the cross-bar extends in a notch in the spacer.

5. The assembly of claim 4 further comprising a device connecting the cross-bar to the spacer.

6. The assembly of claim 5 wherein the latter device is a strap.

7. The assembly of claim 1 further comprising two retainers that receive the cross-bar, and a screw extending through each retainer and into one of the structures to connect the cross-bar to the one structure.

8. The assembly of claim 1 wherein one of the structures is a spinous process.

9. The assembly of claim 8 wherein the spinous process extends from a vertebra of the spine and wherein a vertebra adjacent the first-mentioned vertebra does not have a spinous process, and wherein the spacer stabilizes the spinal column between the two vertebrae.

10. The assembly of claim 9 wherein the cross-bar is connected to one of the latter two vertebrae.

11. The assembly of claim 10 further comprising two retainers that receive the respective ends of the cross-bar, and a screw extending through each retainer and into the one vertebra to connect the cross-bar to the one of the latter two vertebrae.

12. A surgical procedure comprising:
engaging one anatomical structure with a spacer;
connecting the spacer to the one structure; and
securing at least one cross-bar to the spacer and to another anatomical structure.

13. The procedure of claim 12 wherein the step of connecting comprises extending a strap through the spacer and around the one structure.

14. The procedure of claim 12 further comprising extending the cross-bar through an opening in the spacer.

15. The procedure of claim 12 further comprising extending the cross-bar in a notch in the spacer.

16. The procedure of claim 15 further comprising connecting the cross-bar to the spacer.

17. The procedure of claim 15 wherein the latter step of connecting comprises extending a strap around the cross-bar and the spacer.

18. The procedure of claim 12 further comprising extending the cross-bar into two retainers that receive the respective end portions of the cross-bar, and fastening the retainer to the other structure to connect the cross-bar to the one structure.

19. The procedure of claim 12 wherein one of the structures is a spinous process.

20. The procedure of claim 19 wherein the spinous process extends from a vertebra of the spine and wherein a vertebra adjacent the first-mentioned vertebra does not have a spinous process, and wherein the spacer stabilizes the spinal column between the two vertebrae.

21. The procedure of claim 20 wherein the cross-bar is connected to one of the latter two vertebrae.

22. The procedure of claim 21 further comprising extending the respective end portions of the cross-bar into two retainers, and fastening the retainers to the one of the latter two vertebrae.
